(19) United States
(12) Patent Application Publication
(10) Pub. No.: US 2025/0387260 A1
(43) Pub. Date: Dec. 25, 2025

(54) SYSTEMS AND METHODS TREATING FOR CORNEAL ECTATIC DISORDERS

(71) Applicant: Avedro, Inc., Burlington, MA (US)

(72) Inventors: Rajesh K. Rajpal, McLean, VA (US); Grace Elizabeth Lytle, Boston, MA (US)

(21) Appl. No.: 19/310,583

(22) Filed: Aug. 26, 2025

Related U.S. Application Data
(63) Continuation of application No. 16/575,428, filed on Sep. 19, 2019, now Pat. No. 12,453,652.
(60) Provisional application No. 62/733,617, filed on Sep. 19, 2018.

(51) Int. Cl. A61F 9/00; A61F 9/008; A61N 5/06

(52) U.S. Cl. CPC A61F 9/0017; A61N 5/062; A61F 2009/00872; A61F 2009/00893; A61N 2005/0661; A61N 2005/0662; A61N 2005/0665

(57) ABSTRACT

To treat corneal ectatic disorders, systems and methods can precisely apply photoactivating light to specified areas of a cornea treated with a cross-linking agent. An example system includes a light source that provides a photoactivating light to photoactivate a cross-linking agent applied to an eye. The system includes optical element(s) that transmit the photoactivating light to the eye according to a pattern defined by a plurality of treatment zones. The treatment zones are delivered to different respective areas on the eye. The plurality of treatment zones includes at least a first treatment zone and a second treatment zone. The first treatment zone provides a first dose of the photoactivating light. The second treatment zone provides a second dose of the photoactivating light. The first dose is greater than the second dose. The first treatment zone is disposed within an inner boundary of the second treatment zone.

### CROSS-REFERENCE TO RELATED APPLICATIONS

This application is a continuation of U.S. patent application Ser. No. 16/575,428, filed Sep. 19, 2019, titled SYSTEMS AND METHODS TREATING FOR CORNEAL ECTATIC DISORDERS, which claims the benefit of, and priority to, U.S. Provisional Patent Application No. 62/733,617, filed Sep. 19, 2018, the contents of which are incorporated entirely herein by reference.

### BACKGROUND

### Field

The present disclosure pertains to systems and methods for eye treatments, and more particularly, to systems and methods for treating corneal ectatic disorders.

### Description of Related Art

Corneal ectatic disorders, or corneal ectasia, are a group of uncommon, noninflammatory, eye disorders characterised by bilateral thinning of the central, paracentral, or peripheral cornea.

For instance, keratoconus is a degenerative disorder of the eye in which structural changes within the cornea cause it to weaken and change to an abnormal conical shape. Cross-linking treatments can strengthen and stabilize areas weakened by keratoconus and prevent undesired shape changes.

For instance, a complication known as post-LASIK ectasia may occur due to the thinning and weakening of the cornea caused by Laser-Assisted in situ Keratomileusis surgery (LASIK) surgery. In post-LASIK ectasia, the cornea experiences progressive steepening (bulging). Accordingly, cross-linking treatments can strengthen and stabilize the structure of the cornea after LASIK surgery and prevent post-LASIK ectasia.

### SUMMARY

To treat corneal ectatic disorders, such as keratoconus, systems and methods can precisely apply photoactivating light to specified areas of a cornea treated with a cross-linking agent.

An example system for treating an eye includes a light source configured to provide a photoactivating light that photoactivates a cross-linking agent applied to an eye. The system includes one or more optical elements configured to receive the photoactivating light and transmit the photoactivating light to the eye according to a pattern defined by a plurality of treatment zones. The treatment zones are delivered to different respective areas on the eye. The plurality of treatment zones includes at least a first treatment zone and a second treatment zone. The first treatment zone provides a first dose of the photoactivating light. The second treatment zone provides a second dose of the photoactivating light. The first dose is greater than the second dose. The first treatment zone is disposed within an inner boundary of the second treatment zone.

An example method for treating an eye includes determining a location of a treatment area on the eye. The method includes operating at least one of a light source for photoactivating light or one or more optical elements coupled to the light source to deliver a pattern of photoactivating light according to the location of the treatment area. The photoactivating light photoactivates a cross-linking agent applied to the eye. The pattern of photoactivating light is defined by a plurality of treatment zones. The treatment zones are delivered to different respective areas on the eye. The plurality of treatment zones includes at least a first treatment zone and a second treatment zone. The first treatment zone provides a first dose of the photoactivating light. The second treatment zone provides a second dose of the photoactivating light. The first dose is greater than the second dose. The first treatment zone is disposed within an inner boundary of the second treatment zone. In some cases, the treatment area may correspond to an ectatic cone in the cornea, and at least one of the light source or the one or more optical elements is operated to deliver the first treatment zone to the ectatic cone and to deliver the second treatment zone to areas of the cornea outside the ectatic cone. In further cases, the plurality of treatment zones may include a third treatment zone providing a third dose of the photoactivating light, the third dose being greater than the first dose, the third treatment zone being disposed within an inner boundary of the first treatment zone, and at least one of the light source or the one or more optical elements is operated to deliver the first treatment zone and the third treatment zone to the ectatic cone and to deliver the second treatment zone of the photoactivating light to areas of the cornea outside the ectatic cone.

### BRIEF DESCRIPTION OF THE DRAWINGS

FIG. 1 illustrates an example system that delivers a cross-linking agent and photoactivating light to a cornea of an eye in order to generate cross-linking of corneal collagen, according to aspects of the present disclosure.

FIG. 2 illustrates an example pattern of photoactivating light that can be applied to treat corneal ectatic disorders, according to aspects of the present disclosure.

FIG. 3 illustrates an example method for applying the pattern of FIG. 2 to treat corneal ectatic disorders, according to aspects of the present disclosure.

While the present disclosure is susceptible to various modifications and alternative forms, a specific embodiment thereof has been shown by way of example in the drawings and will herein be described in detail. It should be understood, however, that it is not intended to limit the present disclosure to the particular forms disclosed, but on the contrary, the intention is to cover all modifications, equivalents, and alternatives falling within the spirit of the present disclosure.

### DESCRIPTION

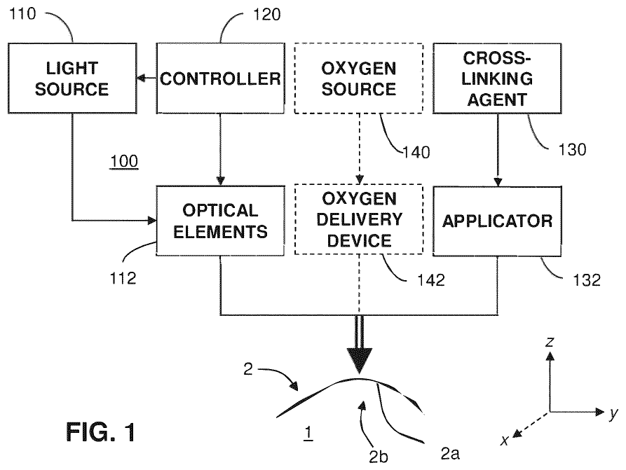

FIG. 1 illustrates an example treatment system 100 for generating cross-linking of collagen in a cornea 2 of an eye 1. The treatment system 100 includes an applicator 132 for applying a cross-linking agent 130 to the cornea 2. In example embodiments, the applicator 132 may be an eye dropper, syringe, or the like that applies the photosensitizer 130 as drops to the cornea 2. Example systems and methods for applying the cross-linking agent are described in U.S. Pat. No. 10,342,697, filed Apr. 13, 2017 and titled “Systems and Methods for Delivering Drugs to an Eye,” the contents of which are incorporated entirely herein by reference.

The cross-linking agent 130 may be provided in a formulation that allows the cross-linking agent 130 to pass through the corneal epithelium 2a and to underlying regions in the corneal stroma 2b. Alternatively, the corneal epithelium 2a may be removed or otherwise incised to allow the cross-linking agent 130 to be applied more directly to the underlying tissue.

The treatment system 100 includes an illumination system with a light source 110 and optical elements 112 for directing light to the cornea 2. The light causes photoactivation of the cross-linking agent 130 to generate cross-linking activity in the cornea 2. For example, the cross-linking agent may include riboflavin and the photoactivating light may include ultraviolet A (UVA) (e.g., approximately 365 nm) light. Alternatively, the photoactivating light may include another wavelength, such as a visible wavelength (e.g., approximately 452 nm). As described further below, corneal cross-linking improves corneal strength by creating chemical bonds within the corneal tissue according to a system of photochemical kinetic reactions. For instance, riboflavin and the photoactivating light may be applied to stabilize and/or strengthen corneal tissue to address corneal ectatic disorders, such as keratoconus or post-LASIK ectasia. Additionally, the application of riboflavin and the photoactivating light may to allow for various amounts of refractive correction, which for instance, may involve combinations of myopia, hyperopia, astigmatism, irregular astigmatism, presbyopia and complex corneal refractive surface corrections due to corneal ectatic disorders as well as other conditions of corneal biomechanical alteration/degeneration, etc.

The treatment system 100 includes one or more controllers 120 that control aspects of the system 100, including the light source 110 and/or the optical elements 112. In an implementation, the cornea 2 can be more broadly treated with the cross-linking agent 130 (e.g., with an eye dropper, syringe, etc.), and the photoactivating light from the light source 110 can be selectively directed to regions of the treated cornea 2 according to a particular pattern.

The optical elements 112 may include one or more mirrors or lenses for directing and focusing the photoactivating light emitted by the light source 110 to a particular pattern on the cornea 2. The optical elements 112 may further include filters for partially blocking wavelengths of light emitted by the light source 110 and for selecting particular wavelengths of light to be directed to the cornea 2 for photoactivating the cross-linking agent 130. In addition, the optical elements 112 may include one or more beam splitters for dividing a beam of light emitted by the light source 110, and may include one or more heat sinks for absorbing light emitted by the light source 110. The optical elements 112 may also accurately and precisely focus the photo-activating light to particular focal planes within the cornea 2, e.g., at a particular depths in the underlying region 2b where cross-linking activity is desired.

Moreover, specific regimes of the photoactivating light can be modulated to achieve a desired degree of cross-linking in the selected regions of the cornea 2. The one or more controllers 120 may be used to control the operation of the light source 110 and/or the optical elements 112 to precisely deliver the photoactivating light according to any combination of: wavelength, bandwidth, intensity, power, location, depth of penetration, and/or duration of treatment (the duration of the exposure cycle, the dark cycle, and the ratio of the exposure cycle to the dark cycle duration).

The parameters for photoactivation of the cross-linking agent 130 can be adjusted, for example, to reduce the amount of time required to achieve the desired cross-linking. In an example implementation, the time can be reduced from minutes to seconds. While some configurations may apply the photoactivating light at an irradiance of 5 mW/cm², larger irradiance of the photoactivating light, e.g., multiples of 5 mW/cm², can be applied to reduce the time required to achieve the desired cross-linking. The total dose of energy absorbed in the cornea 2 can be described as an effective dose, which is an amount of energy absorbed through an area of the corneal epithelium 2a. For example the effective dose for a region of the corneal surface 2A can be, for example, approximately 5 J/cm², or as high as approximately 20 J/cm² or approximately 30 J/cm². The effective dose described can be delivered from a single application of energy, or from repeated applications of energy.

The optical elements 112 of the treatment system 100 may include a microelectromechanical system (MEMS) device, e.g., a digital micro-mirror device (DMD), to modulate the application of photoactivating light spatially and temporally. Using DMD technology, the photoactivating light from the light source 110 is projected in a precise spatial pattern that is created by microscopically small mirrors laid out in an array on a semiconductor chip. Each mirror represents one or more pixels in the pattern of projected light. With the DMD one can perform topography guided cross-linking. The control of the DMD according to topography may employ several different spatial and temporal irradiance and dose profiles. These spatial and temporal dose profiles may be created using continuous wave illumination but may also be modulated via pulsed illumination by pulsing the illumination source under varying frequency and duty cycle regimes. Alternatively, the DMD can modulate different frequencies and duty cycles on a pixel by pixel basis to give ultimate flexibility using continuous wave illumination. Or alternatively, both pulsed illumination and modulated DMD frequency and duty cycle combinations may be combined. This allows for specific amounts of spatially determined corneal cross-linking. This spatially determined cross-linking may be combined with dosimetry, interferometry, optical coherence tomography (OCT), corneal topography, etc., for pre-treatment planning and/or real-time monitoring and modulation of corneal cross-linking during treatment. Aspects of a dosimetry system are described in further detail below. Additionally, pre-clinical patient information may be combined with finite element biomechanical computer modeling to create patient specific pre-treatment plans.

To control aspects of the delivery of the photoactivating light, embodiments may also employ aspects of multiphoton excitation microscopy. In particular, rather than delivering a single photon of a particular wavelength to the cornea 2, the treatment system 100 may deliver multiple photons of longer wavelengths, i.e., lower energy, that combine to initiate the cross-linking. Advantageously, longer wavelengths are scattered within the cornea 2 to a lesser degree than shorter wavelengths, which allows longer wavelengths of light to penetrate the cornea 2 more efficiently than light of shorter wavelengths. Shielding effects of incident irradiation at deeper depths within the cornea are also reduced over conventional short wavelength illumination since the absorption of the light by the photosensitizer is much less at the longer wavelengths. This allows for enhanced control over depth specific cross-linking. For example, in some embodiments, two photons may be employed, where each photon carries approximately half the energy necessary to excite the molecules in the cross-linking agent 130 to generate the photochemical kinetic reactions described further below. When a cross-linking agent molecule simultaneously absorbs both photons, it absorbs enough energy to release reactive radicals in the corneal tissue. Embodiments may also utilize lower energy photons such that a cross-linking agent molecule must simultaneously absorb, for example, three, four, or five, photons to release a reactive radical. The probability of the near-simultaneous absorption of multiple photons is low, so a high flux of excitation photons may be required, and the high flux may be delivered through a femtosecond laser.

A large number of conditions and parameters affect the cross-linking of corneal collagen with the cross-linking agent 130. For example, the irradiance and the dose of photoactivating light affect the amount and the rate of cross-linking.

When the cross-linking agent 130 is riboflavin in particular, the UVA light may be applied continuously (continuous wave (CW)) or as pulsed light, and this selection has an effect on the amount, the rate, and the extent of cross-linking. If the UVA light is applied as pulsed light, the duration of the exposure cycle, the dark cycle, and the ratio of the exposure cycle to the dark cycle duration have an effect on the resulting corneal stiffening. Pulsed light illumination can be used to create greater or lesser stiffening of corneal tissue than may be achieved with continuous wave illumination for the same amount or dose of energy delivered. Light pulses of suitable length and frequency may be used to achieve more optimal chemical amplification. For pulsed light treatment, the on/off duty cycle may be between approximately 1000/1 to approximately 1/1000; the irradiance may be between approximately 1 mW/cm² to approximately 1000 mW/cm² average irradiance, and the pulse rate may be between approximately 0.01 HZ to approximately 1000 Hz or between approximately 1000 Hz to approximately 100,000 Hz.

The treatment system 100 may generate pulsed light by employing a DMD, electronically turning the light source 110 on and off, and/or using a mechanical or opto-electronic (e.g., Pockels cells) shutter or mechanical chopper or rotating aperture. Because of the pixel specific modulation capabilities of the DMD and the subsequent stiffness impartment based on the modulated frequency, duty cycle, irradiance and dose delivered to the cornea, complex biomechanical stiffness patterns may be imparted to the cornea. A specific advantage of the DMD system and method is that it allows for randomized asynchronous pulsed topographic patterning, creating a non-periodic and uniformly appearing illumination which eliminates the possibility for triggering photosensitive epileptic seizures or flicker vertigo for pulsed frequencies between 2 Hz and 84 Hz.

Although example embodiments may employ stepwise on/off pulsed light functions, it is understood that other functions for applying light to the cornea may be employed to achieve similar effects. For example, light may be applied to the cornea according to a sinusoidal function, sawtooth function, or other complex functions or curves, or any combination of functions or curves. Indeed, it is understood that the function may be substantially stepwise where there may be more gradual transitions between on/off values. In addition, it is understood that irradiance does not have to decrease down to a value of zero during the off cycle, and may be above zero during the off cycle. Desired effects may be achieved by applying light to the cornea according to a curve varying irradiance between two or more values.

Examples of systems and methods for delivering photoactivating light are described, for example, in U.S. Patent Application Publication No. 2011/0237999, filed Mar. 18, 2011 and titled “Systems and Methods for Applying and Monitoring Eye Therapy,” U.S. Patent Application Publication No. 2012/0215155, filed Apr. 3, 2012 and titled “Systems and Methods for Applying and Monitoring Eye Therapy,” and U.S. Patent Application Publication No. 2013/0245536, filed Mar. 15, 2013 and titled “Systems and Methods for Corneal Cross-Linking with Pulsed Light,” the contents of these applications being incorporated entirely herein by reference. Embodiments may generate cross-linking activity in the cornea according to circular and/or annular patterns defined by the delivery of photoactivating light (e.g., via the DMD described above). Additionally or alternatively, embodiments may generate cross-linking activity in the cornea according to non-circular and/or non-annular patterns defined by the delivery of photoactivating light (e.g., via the DMD).

Patterns of photoactivating light can be applied (e.g., via the DMD) to the eye in separate treatment zones with different doses sequentially or continuously applied. For instance, one treatment zone can be “turned off” (i.e., delivery of the corresponding photoactivating light ceases) while another “stays on” (i.e., delivery of the corresponding photoactivating light continues). The treatment zones can be, for instance, annularly shaped about a center point of the eye. There may also be discontinuous zones where no the photoactivating light is applied (e.g., a central treatment zone surrounded by an annulus of no light surrounded by an annular treatment zone of light, etc.). The widths of the annular zones can be of different dimensions, e.g., one annular zone has a width of 1 mm and another has a width of 2 mm. Applying the photoactivating light in annular treatment zones on the periphery of the eye without a central treatment zone can result in a hyperopic correction, for instance, by causing the central region of the eye to have an increased curvature while the periphery is strengthened. In some cases, central and surrounding treatment zones can be elliptical in shape, for instance to address astigmatism, by preferentially generating cross-linking activity in regions of the cornea to correct the astigmatism. Such elliptically shaped annular treatment zones are preferentially oriented with the axis of the annular treatment zones aligned according to the orientation of the astigmatism. The elliptically shaped treatment zones can also be irregularly asymmetric (i.e., having major and minor axis that are not perpendicular and can be situated with distinct center points (centers of mass)).

Cross-linking treatments can be tuned according to one or more biomechanical properties of the eye, such as the corneal topography (i.e., shape), corneal strength (i.e., stiffness), and/or corneal thickness. Optical correction and/or strengthening of the cornea can be achieved by applying the cross-linking agent and/or photoactivating light in one or more iterations with adjustable characteristics for each iteration. Generally, a developed treatment plan can include a number of applications of the cross-linking agent, the amount and concentration of the cross-linking agent for each application, the number of applications of photoactivating light, and the timing, duration, power, energy dosage, and pattern of the photoactivating light for each application. Furthermore, the cross-linking treatments can be adapted based on feedback information relating to the biomechanical properties gathered in real-time during treatment or during breaks in treatments.

The addition of oxygen also affects the amount of corneal stiffening. In human tissue, O₂ content is very low compared to the atmosphere. The rate of cross-linking in the cornea, however, is related to the concentration of O₂ when it is irradiated with photoactivating light. Therefore, it may be advantageous to increase or decrease the concentration of O₂ actively during irradiation to control the rate of cross-linking until a desired amount of cross-linking is achieved. Oxygen may be applied during the cross-linking treatments in a number of different ways. One approach involves supersaturating the riboflavin with O₂. Thus, when the riboflavin is applied to the eye, a higher concentration of O₂ is delivered directly into the cornea with the riboflavin and affects the reactions involving O₂ when the riboflavin is exposed to the photoactivating light. According to another approach, a steady state of O₂ (at a selected concentration) may be maintained at the surface of the cornea to expose the cornea to a selected amount of O₂ and cause O₂ to enter the cornea. As shown in FIG. 1, for instance, the treatment system 100 also includes an oxygen source 140 and an oxygen delivery device 142 that optionally delivers oxygen at a selected concentration to the cornea 2. Example systems and methods for applying oxygen during cross-linking treatments are described, for example, in U.S. Pat. No. 8,574,277, filed Oct. 21, 2010 and titled “Eye Therapy,” U.S. Pat. No. 9,707,126, filed Oct. 31, 2012 and titled “Systems and Methods for Corneal Cross-Linking with Pulsed Light,” the contents of these applications being incorporated entirely herein by reference. Additionally, an example mask device for delivering concentrations of oxygen as well as photoactivating light in eye treatments is described in U.S. Provisional Patent Application Publication No. 2017/0156926, filed Dec. 3, 2016 and titled “Systems and Methods for Treating an Eye with a Mask Device,” the contents of which are incorporated entirely herein by reference. For instance, a mask may be placed over the eye(s) to produce a consistent and known oxygen concentration above the surface.

When riboflavin absorbs radiant energy, especially light, it undergoes photoactivation. There are two photochemical kinetic pathways for riboflavin photoactivation, Type I and Type II. The reactions involved in both the Type I and Type II mechanisms and other aspects of the photochemical kinetic reactions generating cross-linking activity are described in U.S. Patent Application Publication No. 10,350,111, filed Apr. 27, 2016 and titled “Systems and Methods for Cross-Linking Treatments of an Eye,” the contents of which are incorporated entirely herein by reference.

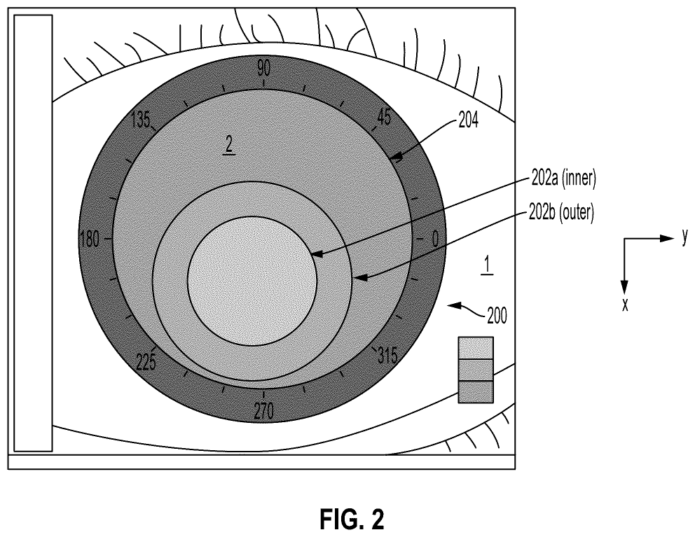

To treat corneal ectatic disorders, such as keratoconus, an effective cross-linking procedure precisely applies photoactivating light to specified areas of a cornea treated with a cross-linking agent. For instance, FIG. 2 illustrates an example pattern 200 of photoactivating light that can be applied to treat corneal ectatic disorders associated with the cornea 2 of the eye 1. As described above, UV light may be delivered according to the pattern 200 to photoactivate a cross-linking agent, such as riboflavin, which has been applied to the cornea 2. As shown, the pattern 200 includes higher-dose treatment zones 202a, b and a lower-dose treatment zone 204. The higher-dose treatment zones 202a, b provide more energy via the photoactivating light than the lower-dose treatment zone 204.

The higher-dose treatment zones 202a, b are centered on and cover the extent of an ectatic cone caused by a disorder, such as keratoconus. As such, the higher-dose treatment zones 202a, b can reduce a curvature of (i.e., flatten) the ectatic cone. The location of the ectatic cone can be determined, for instance, by evaluating the topography, tomography, and/or pachymetry of the cornea 2. As shown in FIG. 2, the higher-dose treatment zone 202a is concentrically disposed within the higher-dose treatment zone 202b. The inner higher-dose treatment zone 202a provides a dose of approximately 10.5 J/cm² and the outer higher-dose treatment zone 202b provides a dose of approximately 8.5 J/cm².

Meanwhile, the lower-dose treatment zone 204 extends from the outer edge of the higher-dose treatment zones 202a, b and covers the surrounding areas of the cornea, where the outer edge of the lower-dose treatment zone 204 does not extend over the limbus. As such, the lower-dose treatment zone 204 stabilizes the surrounding non-ectatic cornea. As shown in FIG. 2, the lower-dose treatment zone 204 is greater than zero and provides a dose of approximately 5.4 J/cm². In some aspects, other approaches teach away from using the lower-dose treatment zone 204 in the pattern 200, as it has been believed that the application of photoactivating light to areas outside the ectatic cone might have an undesired effect on the efficacy of the photoactivating light applied to ectatic cone, e.g., prevent the desired flattening of the ectatic cone.

The shape of the pattern 200 along the x-y plane shown in FIG. 2 can be achieved by adjusting aspects of the optical elements 112 as described above. For instance, the DMD may be programmed via the controller 120 to define the higher-dose treatment zones 202a, b and the lower-dose treatment zone 204 as different respective pixelated shapes applied to the cornea 2. Meanwhile, the depth along the z-axis for delivery of the photoactivating light can be achieved by adjusting the irradiance of the photoactivating light.

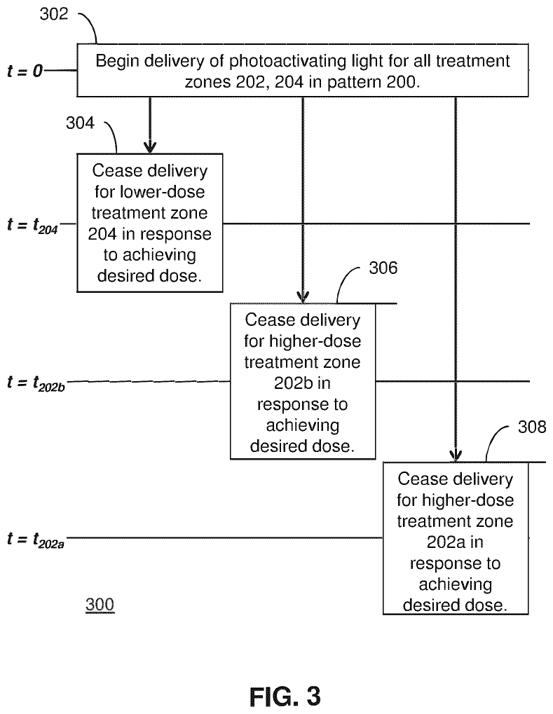

FIG. 3 illustrates an example method 300 for applying the photoactivating light according to the pattern 200. At time t=0, delivery of the photoactivating light for all treatment zones 202a, b, 204 is initiated substantially simultaneously in act 302. At time t=t₂₀₄, delivery of the photoactivating light for the lower treatment zone 204 ceases in act 304 as the desired lower dose, e.g., approximately 5.4 J/cm², is achieved. At time t=t_{202b}, delivery of the photoactivating light for the outer higher-dose treatment zone 202b ceases in subsequent act 306 as the desired higher dose, e.g., approximately 8.5 J/cm² is achieved. At time t=t₂₀₂ₐ, delivery of the photoactivating light for the inner higher-dose treatment zone 202a ceases in subsequent act 308 as the desired higher dose, e.g., approximately 10.5 J/cm² is achieved.

Although the example pattern 200 shown in FIG. 2 may include the two higher-dose treatment zones 202a, b, other patterns may include only one higher-dose treatment zone. (As shown in FIG. 2, for instance, equal doses can be provided in the treatment zones 202a, b.) Alternatively, other patterns may include more than two higher-dose treatment zones. Although the example pattern 200 shown in FIG. 2 may include one lower-dose treatment zone 204, other patterns may include more than one lower-dose treatment zone outside the ectatic cone.

It is also understood that the treatment zones may be located and/or shaped differently than shown in FIG. 2. For instance, the treatment zones may elliptically shaped. Furthermore, although the treatment zones 202a, b, 204 may provide the specified doses described above, other patterns may provide different doses in respective treatment zones. In addition, the relationship of doses between the different treatment zones may be different from those shown in FIG. 2. For instance, the dose(s) provided outside the ectatic cone may be higher than the dose(s) provided for the ectatic cone.

As described above, according to some aspects of the present disclosure, some or all of the steps of the above-described and illustrated procedures can be automated or guided under the control of a controller (e.g., the controller 120). Generally, the controllers may be implemented as a combination of hardware and software elements. The hardware aspects may include combinations of operatively coupled hardware components including microprocessors, logical circuitry, communication/networking ports, digital filters, memory, or logical circuitry. The controller may be adapted to perform operations specified by a computer-executable code, which may be stored on a computer readable medium.

As described above, the controller may be a programmable processing device, such as an external conventional computer or an on-board field programmable gate array (FPGA) or digital signal processor (DSP), that executes software, or stored program instructions. In general, physical processors and/or machines employed by embodiments of the present disclosure for any processing or evaluation may include one or more networked or non-networked general purpose computer systems, microprocessors, field programmable gate arrays (FPGA's), digital signal processors (DSP's), micro-controllers, and the like, programmed according to the teachings of the example embodiments of the present disclosure, as is appreciated by those skilled in the computer and software arts. The physical processors and/or machines may be externally networked with the image capture device(s), or may be integrated to reside within the image capture device. Appropriate software can be readily prepared by programmers of ordinary skill based on the teachings of the example embodiments, as is appreciated by those skilled in the software art. In addition, the devices and subsystems of the example embodiments can be implemented by the preparation of application-specific integrated circuits or by interconnecting an appropriate network of conventional component circuits, as is appreciated by those skilled in the electrical art(s). Thus, the example embodiments are not limited to any specific combination of hardware circuitry and/or software.

Stored on any one or on a combination of computer readable media, the example embodiments of the present disclosure may include software, or stored program instructions, for controlling the devices and subsystems of the example embodiments, for driving the devices and subsystems of the example embodiments, for enabling the devices and subsystems of the example embodiments to interact with a human user, and the like. Such software can include, but is not limited to, device drivers, firmware, operating systems, development tools, applications software, and the like. Such computer readable media further can include the computer program product of an embodiment of the present disclosure for performing all or a portion (if processing is distributed) of the processing performed in implementations. Computer code devices of the example embodiments of the present disclosure can include any suitable interpretable or executable code mechanism, including but not limited to scripts, interpretable programs, dynamic link libraries (DLLs), Java classes and applets, complete executable programs, and the like. Moreover, parts of the processing of the example embodiments of the present disclosure can be distributed for better performance, reliability, cost, and the like.

Common forms of computer-readable media may include, for example, a floppy disk, a flexible disk, hard disk, magnetic tape, any other suitable magnetic medium, a CD-ROM, CDRW, DVD, any other suitable optical medium, punch cards, paper tape, optical mark sheets, any other suitable physical medium with patterns of holes or other optically recognizable indicia, a RAM, a PROM, an EPROM, a FLASH-EPROM, any other suitable memory chip or cartridge, a carrier wave or any other suitable medium from which a computer can read.

While the present disclosure has been described with reference to one or more particular embodiments, those skilled in the art will recognize that many changes may be made thereto without departing from the spirit and scope of the present disclosure. Each of these embodiments and obvious variations thereof is contemplated as falling within the spirit and scope of the present disclosure. It is also contemplated that additional embodiments according to aspects of the present disclosure may combine any number of features from any of the embodiments described herein.

What is claimed is:

1: A method of treating a corneal ectatic disorder of an eye, the method comprising:
evaluating one or more of a topography, a tomography, and a pachymetry of a cornea to determine a location of an ectatic cone of the cornea;
defining, by a controller, a lower-dose treatment zone and a higher-dose treatment zone that includes the location of the ectatic cone;
delivering oxygen by an oxygen delivery device to a surface of the eye;
delivering photoactivating light at a first dose to the higher-dose treatment zone, wherein the controller is programmed to control optical elements to shape the photoactivating light to the higher-dose treatment zone; and
delivering photoactivating light at a second dose to the lower-dose treatment zone, where the controller is programmed to control optical elements to shape the photoactivating light to the lower-dose treatment zone and the second dose is less than the first dose,
wherein the photoactivating light is delivered by a system comprising a photoactivating light source and the optical elements at a frequency determined by one or both of a light pulse rate and an optical elements modulation rate.

2: The method of claim 1, wherein the higher-dose treatment zone and the lower-dose treatment zone do not extend over a limbus of the eye.

3: The method of claim 1, wherein the optical elements are digital micro-mirror devices (“DMDs”).

4: The method of claim 3, wherein the optical elements are configured to produce a pattern defined by the higher-dose treatment zone and the lower-dose treatment zone.

5: The method of claim 1, wherein a pulse rate of the light is between approximately 0.01 Hz to 1,000 Hz.

6: The method of claim 1, wherein a pulse rate of the light is between approximately 1,000 Hz to 100,000 Hz.

7: The method of claim 1, wherein the higher-dose treatment zone and the lower-dose treatment zone are concentric.

8: The method of claim 1, wherein the higher-dose treatment zone and the lower-dose treatment zone are defined by pixels of the photoactivating light.

9: The method of claim 1, wherein the higher-dose treatment zone is disposed within an inner boundary of the lower-dose treatment zone.

10: The method of claim 1, further comprising:
defining, by a controller, a third treatment zone being disposed within an inner boundary of the higher-dose treatment zone.

11: The method of claim 10, wherein the higher-dose treatment zone and the third treatment zone are concentric.

12: The method of claim 10, further comprising:
delivering photoactivating light at a third dose to the third treatment zone, where the controller is programmed to control optical elements to shape the photoactivating light to the third treatment zone

13: The method of claim 12, wherein the third dose is greater than the second dose, and wherein the third dose is less than the first dose.

14: The method of claim 1, further comprising:
simultaneously initiating delivery of the higher-dose treatment zone and the lower-dose treatment zone to the ectatic cone.

15: The method of claim 1, further comprising:
ceasing delivery of the second treatment zone after the second dose has been delivered.

16: The method of claim 1, further comprising:
ceasing delivery of the first treatment zone after the first dose has been delivered, wherein delivery of the second treatment zone ceases before delivery of the first treatment zone ceases.

17: The method of claim 1, further comprising:
applying a cross-linking agent to the eye.

18: The method of claim 1, further comprising:
delivering photoactivating light to one or more depths along an axis extending under a surface of the eye by adjusting an irradiance of the photoactivating light in the higher-dose treatment zone.

19: The method of claim 18, further comprising:
delivering photoactivating light to one or more depths along an axis extending under a surface of the eye by adjusting an irradiance of the photoactivating light in the lower-dose treatment zone.

20: The method of claim 19, wherein a pattern of photoactivating light produced by delivery of photoactivating light is defined along a plane transverse to the axis.
